(19) United States
(12) Patent Application Publication
(10) Pub. No.: US 2016/0209412 A1
(43) Pub. Date: Jul. 21, 2016

(54) MSIA-SRM ASSAY FOR BIOMARKER ANALYSIS

(71) Applicant: NUCLEA BIOTECHNOLOGIES, INC., Pittsfield, MA (US)

(72) Inventors: Sun W. Tam, Westborough, MA (US); Ying Xu, Newton, MA (US); Yunhui Zhang, Shrewsbury, MA (US); Christopher S. Drescher, Frederick, MD (US); Patrick J Muraca, Pittsfield, MA (US)

(73) Assignee: Nuclea Biotechnologies, Inc., Pittsfield, MA (US)

(21) Appl. No.: 14/913,476

(22) PCT Filed: Aug. 20, 2014
(86) PCT No.: PCT/US2014/051835

§ 371 Date: Feb. 22, 2016

(51) Int. Cl. G01N 33/573; H01J 49/00; C07K 7/08; C07K 14/00; C07K 7/06

(52) U.S. Cl. CPC G01N 33/573; C07K 14/001; C07K 7/06; C07K 7/08; H01J 49/0036; G01N 2496/00; G01N 2560/00; G01N 2333/91057

(57) ABSTRACT

The present disclosure provides assays, methods and signature peptides for the identification and quantification of biomarkers in a sample. In particular, the present disclosure relates to the development of mass spectrometric immunoassays with selected reaction monitoring mass spectrometry (MSIA-SRM MS) platforms for biomarker analysis. The MSIA-SRM MS platform may specifically be used for discriminating between particular variants of one or more biomarkers. In addition, the MSIA-SRM platform of the present invention may identify and/or quantify a biomarker and/or its variants present at low abundance in a sample.

### CROSS REFERENCE TO RELATED APPLICATIONS

The present application claims priority of U.S. Provisional Patent Application Ser. No. 61/869,087, filed Aug. 23, 2013, entitled “MSIA-SRM assay for biomarker analysis”, the content of which is by reference incorporated herein in its entirety.

### REFERENCE TO THE SEQUENCE LISTING

The present application is being filed along with a Sequence Listing in electronic format. The Sequence Listing is provided as a file entitled 20151038PCTSEQLST.txt, created on Aug. 20, 2014, which is 66,604 bytes in size. The information in the electronic format of the sequence listing is incorporated herein by reference in its entirety.

### FIELD OF THE INVENTION

The present invention relates to the development of assays and methods for protein analysis by mass spectrometric immunoassay with selected reaction monitoring mass spectrometry (MSIA-SRM). The present invention provides MSIA-SRM platforms for the identification and quantitative measurement of one or more biomarkers of disease. Furthermore, the mass spectrometric immunoassay (MSIA), in combination with other types of mass spectrometry, may also be used to identify new biomarkers and/or their variants in a sample. The present invention further provides improved methods to identify and quantify proteins at physiologically low abundance in a sample.

### BACKGROUND OF THE INVENTION

Biomarkers, in particular protein biomarkers, are increasingly viewed as key adjuvants to drug discovery and development. Biomarkers, which can be used as diagnostic agents, prognostic factors of disease progression and treatment and/or predictors of clinical outcome, provide a significant advance in developing differentiated approaches to individual patient treatments. For example, various biomarkers have been discovered for specific cancers (e.g. breast cancer, prostate cancer), metabolic disorders and immunological diseases.

Immunoaffinity based assays are the mainstay of protein analysis, which are based on antibodies directed against proteins or isoforms of interest. Detection of antibody-protein (i.e. antigen) complexes provides a quantitative measurement of the amount of protein present in a sample. Classic immunoassays, such as radioimmunoassays (RIA), enzyme immunoassays (EIA), enzyme linked immunosorbent assays (ELISA), immunohistochemistry (IHC) and western blots (WB) are widely used for protein biomarker analysis in research and clinical settings. However, developing an immunoassay is often a time-consuming and resource-intensive effort. Moreover, signals from immunoassays usually do not discriminate between different isoforms, modifications, and other variants of specific proteins in samples, which are often causes of disease.

Mass spectrometry (MS) assays are largely developed for proteomic studies. The application and development of MS to identify proteins or peptides separated via liquid phase (e.g. liquid chromatography) separation techniques and/or gel-based separation techniques have led to significant technological advances in protein (e.g. biomarker) analysis. However, application of MS in biomarker analysis is often impeded by the lack of specificity and the non-quantitative nature of such techniques. Many MS variants have been recently developed for more quantitative measurement of protein biomarkers. For example, a MRM-MS (multiple reaction monitoring mass spectrometry) assay can provide impressive quantitative accuracy and dynamic range for determining protein concentration. However, proteins at very low levels of abundance in samples may lie out of the dynamic range of regular MS assays and are often undetectable in samples. In many disease conditions, disease-related proteins, especially particular isoforms, may comprise mutated or modified forms, or may comprise other protein variants that may be present at low levels in samples, making more sensitive methods of quantitative measurement necessary.

Analysis of proteins of low abundance in samples may require that such proteins be concentrated from the sample before analysis. One such method for concentration is immunoenrichment using antibodies specific for the proteins or peptides of interest. The present invention relates to the development of assays and methods which combine antibody-based immunoassays and mass spectrometry platforms. Such hybrid platforms may be applied to the discovery and quantitative measurement of biomarkers for diseases. Moreover, such new assays and methods may be used to detect and quantify a biomarker and/or its variant(s), in particular, one at very low concentration, in a sample.

### SUMMARY OF THE INVENTION

The present invention relates to the development of methods and platforms combining antibody based immunoassay with mass spectrometry for biomarker analysis. The present invention provides methods as well as signature peptide compositions for determining the presence, absence and concentration of one or more biomarkers in a sample. In one embodiment, methods of the present invention may be used to detect variants of a biomarker in a sample, including, but not limited to, splice isoforms, mutated forms and different post-translational modifications. In one aspect, the present invention relates to mass spectrometry immunoassay (MSIA) with selected reaction monitoring/multiple reaction monitoring mass spectrometry (SRM/MRM MS) for quantitatively measuring one or more biomarker in a sample. In another aspect, methods of the present invention may be used to identify and quantify a biomarker at very low concentrations under physiological conditions, in particular, in a disease.

In some embodiments, the mass spectrometric immunoassay (MSIA) uses an antibody-derived immunoassay, which captures a protein of interest in a sample through the binding of the protein (i.e. antigen) to its specific antibody. Antibodies, with high binding affinity and specificity, may be used to concentrate antigens (e.g. biomarkers) in a sample through repeated binding reactions to reach maximal binding capacity. Concentrated protein of interest in the sample may be readily detected and quantified by mass spectrometry.

According to the present invention, certain peptides have been identified using the MSIA-SRM based method, which may be useful in the determination of the presence, absence and/or concentration of the biomarker A2M. These peptides comprise amino acid sequences selected from the group consisting of SEQ ID NO. 3, SEQ ID NO. 4, SEQ ID NO. 5, SEQ ID NO. 6, SEQ ID NO. 7, SEQ ID NO. 8, SEQ ID NO. 9, SEQ ID NO. 10 and SEQ ID NO.11.

According to the present invention, certain peptides have been identified using the MSIA-SRM based method, which may be useful in the determination of the presence, absence and/or concentration of the cancer biomarker FASN. These peptides comprise amino acid sequences selected from the group consisting of SEQ ID NO. 12, SEQ ID NO. 13, SEQ ID NO. 14, SEQ ID NO. 15, SEQ ID NO.16, SEQ ID NO. 17, SEQ ID NO. 18, SEQ ID NO. 19, SEQ ID NO. 20, SEQ ID NO. 21, SEQ ID NO. 22, SEQ ID NO. 23, SEQ ID NO. 24, SEQ ID NO. 25, SEQ ID NO. 26, SEQ ID NO. 27, SEQ ID NO. 28, SEQ ID NO. 29, SEQ ID NO. 30, SEQ ID NO. 31, SEQ ID NO. 32, SEQ ID NO. 33 and SEQ ID NO. 34.

In some embodiments, the present invention provides kits for quantifying a biomarker in a sample. In one aspect, such kits may comprise one, two, three, four, five and more peptides selected from the group consisting of SEQ ID NO. 3, SEQ ID NO. 4, SEQ ID NO. 5, SEQ ID NO. 6, SEQ ID NO. 7, SEQ ID NO. 8, SEQ ID NO. 9, SEQ ID NO. 10 and SEQ ID NO.11. In another aspect, the kits may comprise one, two, three, four, five or more peptides selected from the group consisting of SEQ ID NO. 12, SEQ ID NO. 13, SEQ ID NO. 14, SEQ ID NO. 15, SEQ ID NO.16, SEQ ID NO. 17, SEQ ID NO. 18, SEQ ID NO. 19, SEQ ID NO. 20, SEQ ID NO. 21, SEQ ID NO. 22, SEQ ID NO. 23, SEQ ID NO. 24, SEQ ID NO. 25, SEQ ID NO. 26, SEQ ID NO. 27, SEQ ID NO. 28, SEQ ID NO. 29, SEQ ID NO. 30, SEQ ID NO. 31, SEQ ID NO. 32, SEQ ID NO. 33 and SEQ ID NO. 34.

Kits may further comprise one, two, three, four, five or more peptides with known concentrations selected from the peptides identified in the present invention as listed above. In addition, kits may contain antibodies specifically reactive to a biomarker.

In another embodiment, the present invention provides synthetic peptides with 5, 6, 7, 8, 9, 10, 11, 12, 13, 14, 15, 16, 17, 18, 19, 20, 21, 22 or 23 contiguous amino acids of a peptide selected from the group consisting of SEQ ID NO. 3, SEQ ID NO. 4, SEQ ID NO. 5, SEQ ID NO. 6, SEQ ID NO. 7, SEQ ID NO. 8, SEQ ID NO. 9, SEQ ID NO. 10, SEQ ID NO.11, SEQ ID NO. 12, SEQ ID NO. 13, SEQ ID NO. 14, SEQ ID NO. 15, SEQ ID NO.16, SEQ ID NO. 17, SEQ ID NO. 18, SEQ ID NO. 19, SEQ ID NO. 20, SEQ ID NO. 21, SEQ ID NO. 22, SEQ ID NO. 23, SEQ ID NO. 24, SEQ ID NO. 25, SEQ ID NO. 26, SEQ ID NO. 27, SEQ ID NO. 28, SEQ ID NO. 29, SEQ ID NO. 30, SEQ ID NO. 31, SEQ ID NO. 32, SEQ ID NO. 33 and SEQ ID NO. 34. In addition, the synthetic peptide may have a detectable label.

### BRIEF DESCRIPTION OF THE DRAWINGS

FIG. 1 is an example of the calibration curve from A2M peptide AIGYLNTGYQR (SEQ ID NO. 4) with R2 equal to 0.9.

FIG. 2 is an example of the calibration curve from FASN peptide GYAVLGGER (SEQ ID NO. 15) with R2 equal to 0.98.

### DETAILED DESCRIPTION OF THE INVENTION

The details of embodiments of the invention are set forth in the accompanying description below. Although materials and methods similar or equivalent to those described herein can be used in the practice or testing of the present invention, the preferred materials and methods are now described. Other features, objects and advantages of the invention will be apparent from the description. In the description, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present description will control.

The present invention relates to the development of assays and methods for protein analysis by mass spectrometric immunoassay with selected reaction monitoring MS (MSIA-SRM). In one aspect, the present invention relates to assays and methods for identifying and/or quantifying one or more biomarker(s) in a sample. In another aspect, the present invention provides MSIA-SRM based platforms for developing high-throughput assays and methods for discriminating and/or quantifying protein heterogeneity which is blind in classic biomarker immunoassays. In addition, the present invention provides highly sensitive assays and methods that can detect and quantify protein biomarkers at low levels of abundance in complex biological samples.

The present invention also provides kits and protein signature peptides useful in the methods and assays of the present invention for the quantitative measurement of proteins (e.g. biomarkers) in a sample. These assays and methods are useful in research for the discovery and evaluation of biomarkers, and for clinical diagnosis, prognosis of progression and treatment and prediction of clinical outcome of diseases, including, but not limited to, cancers, metabolic syndrome, heart diseases, neurodegenerative diseases and immunological diseases.

Accordingly, the present invention integrates immunoassays that may be used to develop biological samples by concentrating a protein of interest from such samples, utilizing the specific affinity of an antibody to its antigen, and mass spectrometry that analyzes and quantifies the concentrated protein.

Classic immunoassays, such as radioimmunoassay (RIA), enzyme immunoassay (EIA), enzyme linked immunosorbent assay (ELISA), immunohistochemistry (IHC) and western blots (WB), are among the important methods for the quantitative protein analysis, and widely used for diagnostic/prognostic biomarker analysis. However, Development of immunoassays often requires a time-consuming and resource-intensive effort. Furthermore, the detection methods and labels used in these antibody-based immunoassays often cannot discriminate between protein isoforms, modifications, mutations and/or other variants thereof, due to the fact that the resulting quantitative signals are often the sum of signals from all forms of a given protein in the sample tested.

Mass spectrometry (MS) based assays have been widely used for detecting protein biomarkers associated with various diseases. See e.g. U.S. Pat. No. 8,440,409, U.S. Pat. No. 8,465,929, U.S. Pat. No. 8,043,825, U.S. Pat. No. 7,935,921, U.S. Pat. No. 7,811,772, U.S. Pat. No. 7,604,948, U.S. Pat. No. 7,115,378, and U.S. Pat. No. 6,177,266, the contents of each of which are herein incorporated by reference in their entirety. The direct sequence identification of different peptides using mass spectrometry is very useful in discriminating between spliced isoforms, modifications, mutations and/or other variants of a protein. Moreover, variants of MS platforms have been developed in the art for more quantitative measurement of biomarkers in samples, such as selected reaction monitoring/multiple reaction monitoring mass spectrometry (SRM/MRM-MS) (see e.g. U.S. Pat. No. 8,383,417, PCT patent application publication No. WO2013106603, U.S. patent application publication No. US2013105684, the contents of each of which are herein incorporated by reference in their entirety.). SRM/MRM methods can combine the high selectivity of MS for proteins of interest (e.g. sliced isoforms of a protein) with quantitative accuracy and dynamic range. Quantitation obtained by SRM/MRM MS methods is based on the peak area for the mass spectra data of an isotope-labeled standard (e.g. a standard signature peptide), which can be used to provide relative quantitation or absolute protein concentration (Gerber et al., Proc. Natl. Asso. Sci., 2003, 100, 6940-6945, the contents of which are herein incorporated by reference in its entirety.). However, proteins at low levels of abundance in samples may need to be concentrated prior to SRM/MRM mass spectrometry.

Immunoaffinity based methods for capturing proteins or peptides of interest in samples, and for developing samples concentrated with such proteins are available in the art, such as for example, a SISCAPA (stable isotope standards and capture by anti-peptide antibodies) method for specific antibody-based capture of individual tryptic peptides from a digest of a sample (e.g. human plasma) (Anderson et al., J. Proteome Research, 2004, 3, 235-244, herein incorporated by reference in its entirety.). Other enrichment methods used in the art include immuno adsorption-based depletion of abundant protein species from samples, immunoprecipitation, affinity ligand binding, bead or membrane based immunoaffinity, etc.

Nelson R W and his coworkers first developed a mass spectrometric immunoassay (MSIA) using affinity pipette tips to selectively retrieve proteins from biological solutions, demonstrating high-throughput quantitative protein analysis (Nelson R W et al., Analytical Chemistry, 1995, 67, 1153-1158, herein incorporated by reference in its entirety). The MSIA platform relies on a pipette immunoenrichment technology that uses a high-capacity micro column (i.e. tips of pipettes) activated with antibodies to isolate low abundance proteins in complex samples for mass spectrometry. See, e.g. U.S. Pat. No. 8,486,713; U.S. Pat. No. 7,399,641; U.S. Pat. No. 7,303,888, U.S. Pat. No. 6,974,704; U.S. Pat. No. 4,022,876, the contents of each of which are herein incorporated by reference in their entirety. MSIA with selected reaction monitoring mass spectrometry (SRM-MS) further enables the quantitative measurement of enriched proteins and variants thereof in a sample. For example, Lopez et al, using the MSIA-SRM method, identified a number of new parathyroid hormone (PTH) variants associated with the hormone that may be useful in developing biomarkers for various skeletal and endocrine diseases (U.S. Pat. No. 838,341; Lopez M F et al., Clinical Chemistry, 2010, 56, 281-290; the contents of each of which are herein incorporated by reference in their entirety). Other references using MSIA-SRM for quantitative protein analysis include the references by Krastins B et al., Clin Biochem, 2013, 46, 399-410; Yassine H et al., Proteomics Clin Appli. 2013, 7, 528-540; U.S. patent application publication No. 2003/0027216; U.S. patent application publication No. 2009/0197284; U.S. Pat. No. 7,396,687; PCT patent application publication No. WO2010071788; the contents of each of which are herein incorporated by reference in their entirety.

In some embodiments, methods of the present invention utilize immunoenrichment techniques. As used herein, the term “immunoenrichment” refers to antibody or antigen-based methods of concentrating one or more proteins of interest. Some immunoenrichment methods comprise column-based immunoenrichment techniques to selectively isolate proteins of interest and elute them to generate more concentrated samples for mass spectrometric analysis. As used herein, the term “column-based immunoenrichment” refers to an immunoenrichment method wherein an antibody or antigen-lined column (referred to herein as an “affinity column”) is used to isolate one or more proteins of interest from a sample, and wherein such proteins are subsequently eluted, creating a sample wherein such proteins are more concentrated or “enriched.” Affinity columns utilized in column-based immunoenrichment may comprise low volume columns, referred to herein as “affinity micro columns.” Some affinity columns may comprise pipette tips, referred to herein as “affinity tips.” Column-based immunoenrichment comprising affinity tips is referred to herein as “pipette immuno enrichment.”

In certain embodiments of the present invention, the methods for determining the concentration of a protein biomarker, and/or the variants thereof comprise the steps of: 1) obtaining a biological sample from a subject; 2) concentrating/enriching a protein and its variants of interest from the sample, wherein the step of concentrating the protein of interest comprises capturing the protein and its variants of interest in the sample by column-based immunoenrichment (e.g. micro column-based immunoenrichment), eluting the captured protein and/or its variants from the antibodies and proteolytically digesting the eluted protein and/or its variants; 3) analyzing the digested protein by mass spectrometry and generating a mass spectrometric profile; 4) identifying and/or quantifying the protein and/or its variants of interest (e.g. a biomarker) in the sample.

In some embodiments, column-based immunoenrichment steps of the present methods may comprise pipette immunoenrichment utilizing antibody-derived affinity tips. Antibodies comprised therein may be specific to one or more proteins of interest in a biological sample. Such antibodies may be commercially available from any commercial distributors. For example, the antibodies may be purchased from Abcam (Cambridge, Mass.), Cell Singaling (Danvers, Mass.), DAKO (Carpinteria, Calif.), Life Technologies (Beverly, Mass.), Sigma-Aldrich (St. Louis, Mo.), New England BioLabs (Ipswich, Mass.), R&D systems (Minneapolis, Minn.), Santa Cruz Biotechnology (Dallas, Tex.) and other companies. Antibodies may be monoclonal antibodies or polyclononal antibodies. The antibodies may also be raised in different hosts, including but not limited to mouse, rat, rabbit, sheep and pig hosts. Furthermore, antibodies may be generated against one or more short peptides of a protein of interest, or the full-length protein of interest. Accordingly, antibodies used in the present invention may be specifically reactive to one or more proteins of interest with a high affinity and specificity. For example, the antibody binding to its antigen may have an association rate constant (Ka) of 10⁷ to 10⁹, particularly for the MSIA assay. In addition to classic antibodies, antibody fragments, F(ab)2, Fab, nanobodies may also be used to capture target proteins. Antibodies used in the present invention could be any immunoglobulin isotype from five human immunoglobulin isotypes of IgG, IgM, IgA, IgD and IgE.

According to some embodiments of the present methods, anitbodies may be conjugated or immobilized to a substrate, such as the resin of a pippete tip. Any commercially available substrates may be used for antibody immobilization. As a non-limiting example, the Thermo Scientific MSIA pipette tips may be used to directly immobilize antibodies to the affinity support surface of the pipette tips (Thermo Scientific, Waltham, Mass.). Alternatively, antibodies may be indirectly coupled to affinity support surfaces through a universal ligand, such as the Protein A/Protein G coated tips from Thermo Scientific (Thermo Scientific, Waltham, Mass.). Other universal binding agents may also be used to conjugate antibodies, for example, Streptavidin.

In some embodiments, antibody-derived affinity tips may be made according to any available methods used for antibody conjugation in the art. As a non-limiting example, antibodies may be diluted in a solution (e.g. PBS buffer) at various concentrations. Coupling reactions may be performed by repetitively running antibody solutions over tips for multiple cycles (e.g. 100-500 cycles) to reach the maximum capacity. Each of such cycles may consist of a single aspiration and dispensation through the affinity tips.

In one aspect, antibody conjugated affinity tips may be used to capture a protein and its variants of interest in a biological sample. Any methods for forming antibody-antigen complexes may be used according to the present method. The affinity binding of antibodies to specific antigens (e.g. the protein and its variants of interest) may enrich the protein to be analyzed.

In some embodiments of the present invention, the protein to be analyzed or protein of interest may be a native endogenous protein (or its variant) in a biological sample, a recombinant protein from in vitro translation and/or an isotope labeled protein or polypeptide. While samples may include any sample which is amendable for protein analysis, samples are most often bodily fluid samples, such as serum, plasma, urine, cerebrospinal fluid, etc. Samples may also be obtained from any other protein-containing specimens, such as tissue/cell lysate, biopsies, and cell-culture media. Some samples may be obtained from a subject. As used herein, a “subject” refers to a vertebrate, including, but not limited to mammals. Such mammals may include, but are not limited to primates. Such primates may include, but are not limited to humans.

Some samples may be obtained from subjects who are patients. As used herein, “patient” refers to a subject who may seek or be in need of treatment, requires treatment, is receiving treatment, will receive treatment, or a subject who is under care by a trained professional for a particular disease or condition.

As used herein, the term “sample” or “biological sample” refers to a subset of its tissues, cells or component parts (e.g. body fluids, including but not limited to blood, mucus, lymphatic fluid, synovial fluid, cerebrospinal fluid, saliva, amniotic fluid, amniotic cord blood, urine, vaginal fluid, bone marrow, sputum and semen). A sample further may include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. A sample further refers to a medium, such as a nutrient broth or gel, which may contain cellular components, such as proteins or nucleic acid molecules. Biological samples may also include sections of tissues such frozen sections taken for histological purposes. A biological sample may also be referred to as a “patient sample.” Samples that comprise an elevated concentration of a protein in relation to a starting or reference sample, may be referred to herein as a “concentrated sample” or an “enriched sample.”

In one embodiment, the concentration of a protein of interest (e.g. a biomarker) in a biological sample may be within the pg/ml to ng/ml range. As described herein, the immunoenrichment methods associated with the MSIA platform may be used to concentrate the amount one or more proteins of interest in an original sample such that the concentration of such proteins falls within the detection limit of mass spectrometry allowing for their detection in this physiological range. Some such methods may comprise the use of antibody-based immunoenrichment methods to concentrate such target proteins present at very low abundance in samples.

A biological sample often contains different forms of a protein of interest, such as closely related isoforms (e.g. from alternative splicing,) mutants, different post-translationally modified forms and/or other derivatives. The presence, absence or differential expression of each variant of proteins of interest may be associated with disease in many cases. Thus, it is critically important to discriminate among closely related variants of specific proteins and determine the concentration of such variants within samples. In some embodiments, methods of the present invention using MSIA-SRM assay may capture different variants of a protein of interest and quantitatively measure the amount of one or more such variants in a sample.

In other embodiments, assays and methods of the present invention may be used to detect and/or quantify a recombinant protein or polypeptide expressed in in vitro systems, for example, in E. coli bacteria or cell lines. Such methods may be used to confirm the amino acid sequence of recombinant proteins or polypeptides, identify modifications or mutations of proteins, and determine the concentration of proteins.

In some embodiments, proteins of interest concentrated by antibody-based immunoenrichment methods may be subjected to enzyme digestion to generate short peptides for mass spectrometry analysis. Enzyme digestion may be optimized to minimize the variations between assays and different samples. As used herein, the term “digest” means to break apart into shorter peptides. Such enzymes may include, but are not limited to, trypsin, endoproteinase Glu-C and chymotrypsin.

Mass spectrometry analysis of concentrated and digested protein fragments may be performed using a mass spectrometer which includes an ion source for ionizing fractionated samples and creating charged molecules for further analysis. For example ionization of samples may be performed by electrospray ionization (ESI), atmospheric pressure chemical ionization (APCI), photoionization, electron ionization, fast atom bombardment (FAB)/liquid secondary ionization (LSIMS), matrix assisted laser desorption ionization (MALDI), field ionization, field desorption, thermospray/plasmaspray ionization, and particle beam ionization. The skilled artisan will understand that the choice of ionization method may be determined based on the analyte to be measured, type of sample, the type of detector, the choice of positive versus negative mode, etc.

In some embodiments, ions in ionized samples may be detected using several different detection methods. For example, ions may be detected using selected ion monitoring (SIM), or alternatively using a scanning mode, e.g. selected reaction monitoring (SRM) and/or its variants, such as multiple reaction monitoring (MRM).

In some embodiments, assays and methods of the present invention comprise MSIA with selected reaction monitoring mass spectrometry (MSIA-SRM). Liquid chromatography-selected reaction monitoring mass spectrometry (LC/SRM-MS) methods are peptide sequence-based modes of MS that restrict detection and fragmentation to only those peptides derived from proteins of interest. Such targeted MS methods dramatically improve the sensitivity and reproducibility compared to discovery mode MS methods. The method of MSIA-SRM quantification of proteins can dramatically impact the discovery and quantification of biomarkers via rapid, targeted, multiplexed biomarker analysis in clinical samples.

As used herein, “selected reaction monitoring (SRM)” is a tandem mass spectrometry mode, in which an ion of a particular mass is selected in the first stage of tandem mass spectrometry and an ion product of a fragmentation reaction of the precursor ion is selected in the second mass spectrometry stage for detection. “Multiple reaction monitoring (MRM)” is a method for applying selected reaction monitoring (SRM) to multiple product ions from one or more precursor ions.

Specifically, MSIA-SRM MS allows high-throughput assays to be carried out with high accuracy. In some embodiments, MSIA-SRM methods of the present invention may be used to screen antibody pools for antibodies that bind to an antigen of interest and/or one or more variants thereof. Such antibodies may recognize different variant(s) of the same antigen, including but not limited to post-translational modification(s), related isoforms from alternative splicing, and/or mutated forms of the same antigen due to transcriptional and translational mutation events in disease states, and/or protein complex formation of the antigen with other proteins or metabolites in physiological conditions. Additionally, MSIA assays may be followed by other types of mass spectrometry to identify one or more new biomarker(s) in a sample under certain physiological conditions. As a non-limiting example, MSIA, followed by Quadrupole time of flight (QTOF) mass spectrometry or Orbitrap mass spectrometry may be used to discover one or more new biomarker(s) in a sample.

In some instances, serial immunoassays may be performed in single MSIA-SRM assays. Accordingly, one or more different antibodies may be specifically reactive to one or more proteins or antigens that may be immobilized to the affinity tips. A sample with a heterogeneous population of proteins may be applied to multiple antibody-based affinity tips in serial reactions to allow each protein to be captured by its specific antibody.

In some aspects, methods may be semi-automated or automated. As a non-limiting example, the process may be performed using a Thermo Scientific MSIA plastform from Thermo Scientific which may comprise the Thermo Scientific VERSETTE™ Pipetting Workstation, highly sensitive Thermo Scientific TSQ VANTAGE™ Triple Stage Quadruple Mass Spectrometer, Thermo Scientific Q EXACTIVE™ Hybrid Quadruple-Orbitrap Mass Spectrometer, Thermo Scienific DIONEX™ ULTIMATE™ 3000 RSLCnano Systems and/or PINPOINT™ software (Thermo Scientific, Waltham, Mass.).

In some embodiments, one or more peptides or signature peptides for a protein of interest may be identified by the MSIA-SRM methods of the present invention. Such peptides or signature peptides may be used to generate a standard calibration curve for the determination of the concentration of a protein of interest in a sample.

As used herein, a “standard calibration curve” refers to a curve or table for determining the concentration of a substance (e.g. protein) in a sample by comparing the unknown protein to a set of standard peptides of known concentration. In general, concentrations of standard peptides lie across the range of expected concentrations for proteins of interest in samples. Some signature peptides may be mixed with a sample as internal standards.

As used herein, the term “internal standard” refers to one or more proteins or signature peptides for one or more proteins that are added in a constant amount to a biological sample from a subject in a MSIA-SRM assay. The peptides are then used for calibration by plotting the ratio of the analyte signal to the internal standard signal as a function of analyte concentration of the standards. The peptides used as internal standards are peptides for a protein to be analyzed, for example, a biomarker for a clinical condition.

Specifically, the present MSIA-SRM MS may determine the concentration of a protein and/or all isoforms of said protein, if present in a sample, by comparing signals of said protein in the sample with the standard calibration curve created with the peptides or peptide signatures for said protein. The quantification is based on the relative intensity of the analyte signal, compared to the signal of known levels of internal standards.

Specifically, one or more standard peptides or proteins may be further labeled with a detectable agent, including, but not limited to, a fluorescent label (such as cyanine, fluorescein, rhodamine, sulforhodamine B, tetramethylrhodamine, coumarin, eosin, ATTO dyes, BODIPY dyes, etc.), heavy isotope (such as nitrogen-15, carbon-13, hydrogen-2, sulfur-34, oxygen-18, oxygen-17, etc.) and/or deuterium.

In addition, one or more standard peptides or proteins may be synthesized with any method known in the pertinent art. Such synthetic peptides or proteins may further comprise amino acids with one or more natural modifications. Such natural modifications may include, but are not limited to, deamination of glutamine and asparagine, amination, oxidation and hydroxylation, etc.

### Signature Peptides for FASN

Fatty acid synthase (FASN) plays an important role in an anabolic-energy-storage pathway and lipogenesis. Previous reports have shown that FASN is associated with clinically aggressive tumor behavior, tumor-cell growth and survival. Human cancer cells and certain types of tumors, such as prostate cancer and breast cancer, express higher levels of FASN. The levels of FASN as well as its affiliated molecular targets therefore represent valuable biomarkers in the identification of patient populations that would benefit most from a FASN directed treatment in oncology. However, the concentrations of FASN in clinical samples are typically in the ng/ml range and a very sensitive assay is necessary for quantification.

In one embodiment, MSIA-SRM is applied and optimized to quantify FASN from human samples. The methods of the present invention allow FASN to be tested in biological samples in a high-throughput and semi-automated manner. In one aspect, the methods of the present invention may be used to discover and quantify any isoforms, modifications and/or other variants of FASN (GenBank NM_004104, SEQ ID NO.1) in a sample from a subject. Such subjects may be afflicted with or at risk of developing a disease associated with abnormal FASN levels and/or activity. Such diseases may include, but are not limited breast cancer and/or prostate cancer.

According to the present invention, the signature peptides for FASN were identified with a MSIA-SRM based platform, comprising SEQ ID NO. 12, SEQ ID NO. 13, SEQ ID NO. 14, SEQ ID NO. 15, SEQ ID NO.16, SEQ ID NO. 17, SEQ ID NO. 18, SEQ ID NO. 19, SEQ ID NO. 20, SEQ ID NO. 21, SEQ ID NO. 22, SEQ ID NO. 23, SEQ ID NO. 24, SEQ ID NO. 25, SEQ ID NO. 26, SEQ ID NO. 27, SEQ ID NO. 28, SEQ ID NO. 29, SEQ ID NO. 30, SEQ ID NO. 31, SEQ ID NO. 32, SEQ ID NO. 33 and SEQ ID NO. 34.

In one aspect of the invention, the methods are provided with a standard calibration curve to calculate and determine the concentration of one or more variants of FASN in a sample from a subject. A standard calibration curve may be created using one or more peptides selected from the group consisting of: SEQ ID NO. 12, SEQ ID NO. 13, SEQ ID NO. 14, SEQ ID NO. 15, SEQ ID NO.16, SEQ ID NO. 17, SEQ ID NO. 18, SEQ ID NO. 19, SEQ ID NO. 20, SEQ ID NO. 21, SEQ ID NO. 22, SEQ ID NO. 23, SEQ ID NO. 24, SEQ ID NO. 25, SEQ ID NO. 26, SEQ ID NO. 27, SEQ ID NO. 28, SEQ ID NO. 29, SEQ ID NO. 30, SEQ ID NO. 31, SEQ ID NO. 32, SEQ ID NO. 33 and SEQ ID NO. 34.

In a further aspect, a series of peptides across a range of concentrations from 1.25 ng/mL to 40 ng/mL are prepared. The signal of a set of peptides with known concentration is measured with MRM-MS assay. A standard calibration curve is created by plotting the changes of the analytic signal with the known concentrations of peptides.

According to the present invention, FASN protein and/or FASN variants in a sample obtained from a subject may be concentrated by anti-FASN antibody-based immunoenrichment methods. The concentration of FASN or FASN variants contained in the concentrated sample may then be determined by subjecting the concentrated proteins in the sample to digestion. After digestion, the sample may be analyzed by SRM/MRM MS to generate a mass spectrometric profile. The mass spectrometric profile of the digested sample may then be compared to a standard calibration curve to calculate the concentration of FASN and/or FASN variants in the sample.

In some embodiment of the present invention, synthetic peptides 6-13 amino acids in length are provided. Such synthetic peptides may be used to generate a standard calibration curve. Synthetic peptides, in particular, may have at least 5 contiguous amino acids of a peptide selected from the group consisting of SEQ ID NO. 12, SEQ ID NO. 13, SEQ ID NO. 14, SEQ ID NO. 15, SEQ ID NO.16, SEQ ID NO. 17, SEQ ID NO. 18, SEQ ID NO. 19, SEQ ID NO. 20, SEQ ID NO. 21, SEQ ID NO. 22, SEQ ID NO. 23, SEQ ID NO. 24, SEQ ID NO. 25, SEQ ID NO. 26, SEQ ID NO. 27, SEQ ID NO. 28, SEQ ID NO. 29, SEQ ID NO. 30, SEQ ID NO. 31, SEQ ID NO. 32, SEQ ID NO. 33 and SEQ ID NO. 34. Specifically, such synthetic peptides may be 5, 6, 7, 8, 9, 10, 11, 12 or 13 amino acids in length. Synthetic peptides may further be incorporated with one or more detectable agent, including, but not limited to, a fluorescent label (such as cyanine, fluorescein, rhodamine, sulforhodamine B, tetramethylrhodamine, coumarin, eosin, ATTO dyes, BODIPY dyes, etc), heavy isotope (such as nitrogen-15, carbon-13, hydrogen-2, sulfur-34, oxygen-18, oxygen-17, etc) and/or deuterium. Synthetic peptides may further comprise amino acids with one or more natural modification.

### Signature Peptides for A2M

Alpha-2-Macroglobulin, also known as a2-macroglobulin and abbreviated as a2M and A2M, is a large plasma protein found in the blood. A2M acts as an in vivo protease inhibitor and is able to bind an enormous variety of proteinases, such as plasmin, thrombin and kallikrein. Due to its large size and rapid accumulation in blood, A2M is a supplementary diagnostic marker in nephrotic syndrome, liver cirrhosis and diabetes. A2M has also been recognized as an amyloid plaque diffuser in the brains of Alzheimer's disease (AD) patients, therefore playing an important role in AD etiology.

In some embodiments, MSIA-SRM may be applied and optimized to quantify A2M from human samples. The methods of the present invention allow A2M to be tested in biological samples in a high-throughput and semi-automated manner. In one aspect, the methods of the present invention may be used to discover and quantify any isoforms, modifications and/or variants of A2M (GenBank NM_000014, SEQ ID NO.2) in samples from subjects. Such subjects may be afflicted with or at risk of developing a disease associated with abnormal A2M levels and/or activity, Such diseases may include, but are not limited to diabetes and/or Alzheimer's disease (AD).

According to the present invention, signature peptides for A2M are provided that have been identified with a MSIA-SRM based platform. Such signature peptides include SEQ ID NO. 3, SEQ ID NO. 4, SEQ ID NO. 5, SEQ ID NO. 6, SEQ ID NO. 7, SEQ ID NO. 8, SEQ ID NO. 9, SEQ ID NO. 10 and SEQ ID NO.11.

In one aspect of the invention, the methods are provided with a standard calibration curve to calculate and determine the concentration of one or more variants of A2M in a sample from a subject. A standard calibration curve may be created using one or more peptides selected from the group consisting of: SEQ ID NO. 3, SEQ ID NO. 4, SEQ ID NO. 5, SEQ ID NO. 6, SEQ ID NO. 7, SEQ ID NO. 8, SEQ ID NO. 9, SEQ ID NO. 10 and SEQ ID NO.11.

In a further aspect, a series of peptides across a range of concentrations from 1.25 μg/mL to 15 μg/mL are prepared. Accordingly, the signal of a set of peptides with known concentration is measured by MRM-MS assay and a standard calibration curve is created by plotting the changes of the analytic signal with the known concentration of peptides.

According to the present invention, A2M protein or variants of A2M in a sample obtained from a subject may be concentrated by anti-A2M antibody-based immunoenrichment. The concentration of A2M or variants of A2M contained in the concentrated sample may then be determined by digesting the concentrated A2M or variants of A2M followed by SRM/MRM MS analysis to generate a mass spectrometric profile. The mass spectrometric profile of the digested proteins may then be compared to a standard calibration curve to calculate the concentration of A2M and/or A2M variants in the sample.

According to the present invention, synthetic peptides 6-23 amino acids in length are provided. Such synthetic peptides may be used in the generation of a standard calibration curve. Synthetic peptides may comprise at least 5 contiguous amino acids of a peptide selected from the group consisting of SEQ ID NO. 3, SEQ ID NO. 4, SEQ ID NO. 5, SEQ ID NO. 6, SEQ ID NO. 7, SEQ ID NO. 8, SEQ ID NO. 9, SEQ ID NO. 10 and SEQ ID NO.11. Furthermore, such synthetic peptides may be 5, 6, 7, 8, 9, 10, 11, 12, 13, 14, 15, 16, 17, 18, 19, 20, 21, 22 or 23 amino acids in length. Synthetic peptides may further be incorporated with one or more detectable agents, including, but not limited to, a fluorescent label (such as cyanine, fluorescein, rhodamine, sulforhodamine B, tetramethylrhodamine, coumarin, eosin, ATTO dyes, BODIPY dyes, etc), heavy isotope (such as nitrogen-15, carbon-13, hydrogen-2, sulfur-34, oxygen-18, oxygen-17, etc) and/or deuterium. Synthetic peptides may further comprise amino acids with one or more natural modification.

The peptides or signature peptides identified by methods of the present invention are suited for preparation of kits produced by well-known procedures in the art. The present invention thus provides kits comprising two or more calibration standards, which may be used to quantify the concentration of one or more biomarkers in a sample from a subject. In addition, the calibration standards may be synthetic peptides 6 to 22 amino acids in length with at least 5 contiguous amino acids of a peptide. In one aspect of the invention, the kits for FASN detection may comprise two or more calibration standards selected from the group of peptides consisting of SEQ ID NO. 12, SEQ ID NO. 13, SEQ ID NO. 14, SEQ ID NO. 15, SEQ ID NO.16, SEQ ID NO. 17, SEQ ID NO. 18, SEQ ID NO. 19, SEQ ID NO. 20, SEQ ID NO. 21, SEQ ID NO. 22, SEQ ID NO. 23, SEQ ID NO. 24, SEQ ID NO. 25, SEQ ID NO. 26, SEQ ID NO. 27, SEQ ID NO. 28, SEQ ID NO. 29, SEQ ID NO. 30, SEQ ID NO. 31, SEQ ID NO. 32, SEQ ID NO. 33 and SEQ ID NO. 34. In another aspect of the invention, the kits for A2M detection may comprise two or more calibration standards selected from the group of peptides consisting of SEQ ID NO. 3, SEQ ID NO. 4, SEQ ID NO. 5, SEQ ID NO. 6, SEQ ID NO. 7, SEQ ID NO. 8, SEQ ID NO. 9, SEQ ID NO. 10 and SEQ ID NO.11.

In another aspect of the invention, kits may comprise two or more calibration standard peptides that may be further labeled with a detectable reagent. Such detectable reagents may include, but are not limited to a fluorescent label (such as cyanine, fluorescein, rhodamine, sulforhodamine B, tetramethylrhodamine, coumarin, eosin, ATTO dyes, BODIPY dyes, etc), heavy isotope (such as nitrogen-15, carbon-13, hydrogen-2, sulfur-34, oxygen-18, oxygen-17, etc.) and/or deuterium.

In addition, kits may comprise two or more calibration standards in at least three different concentrations across the range of the expected concentration of proteins of interest in a sample from a subject.

In some embodiments, kits may further comprise antibodies specifically reactive to a protein of interest. Such antibodies may have high binding affinity and/or specificity for such proteins of interest.

Kits may optionally comprise reagents with an identifying description or label or instructions relating to their use in the methods of the present invention. In addition, kits may comprise one or more enzymes to digest proteins in a sample from a subject. The enzymes may include, but are not limited to, trypsin, endoproteinase Glu-C and chymotrypsin.

### DEFINITIONS

For convenience, the meaning of certain terms and phrases employed in the specification, examples, and appended claims are provided below. The definitions are not meant to be limiting in nature and serve to provide a clearer understanding of certain aspects of the present invention.

The term “antibody” refers to a polypeptide ligand substantially encoded by an immunoglobulin gene or immunoglobulin genes, or fragments thereof, whether natural or partially or wholly synthetically produced. An antibody specifically (or selectively) binds and recognizes an epitope (e.g., an antigen). All derivatives thereof that maintain specific binding ability are also included in the term. The term also covers any protein having a binding domain that is homologous or largely homologous to an immunoglobulin binding domain. An antibody may be monoclonal or polyclonal. The antibody may be a member of any immunoglobulin class, including any of the human classes: IgG, IgM, IgA, IgD, and IgE, etc. The recognized immunoglobulin genes include the kappa and lambda light chain constant region genes, the alpha, gamma, delta, epsilon and mu heavy chain constant region genes, and the myriad immunoglobulin variable region genes. “Fc” portion of an antibody refers to that portion of an immunoglobulin heavy chain that comprises one or more heavy chain constant region domains, CH1, CH2 and CH3, but does not include the heavy chain variable region.

The term “antibody fragment” refers to any derivative or portion of an antibody that is less than full-length. In one aspect, the antibody fragment retains at least a significant portion of the full-length antibody's specific binding ability, specifically, as a binding partner. Examples of antibody fragments include, but are not limited to, Fab, Fab′, F(ab′)2, scFv, Fv, dsFv diabody, and Fd fragments. The antibody fragment may be produced by any means. For example, the antibody fragment may be enzymatically or chemically produced by fragmentation of an intact antibody or it may be recombinantly produced from a gene encoding the partial antibody sequence. Alternatively, the antibody fragment may be wholly or partially synthetically produced. The antibody fragment may comprise a single chain antibody fragment. In another embodiment, the fragment may comprise multiple chains that are linked together, for example, by disulfide linkages. The fragment may also comprise a multimolecular complex. A functional antibody fragment may typically comprise at least about 50 amino acids and more typically will comprise at least about 200 amino acids.

The term “monoclonal antibody” as used herein refers to an antibody obtained from a population of substantially homogeneous antibodies, i.e., the individual antibodies comprising the population are identical and/or bind the same epitope, except for possible variants that may arise during production of the monoclonal antibody, such variants generally being present in minor amounts. In contrast to polyclonal antibody preparations that typically include different antibodies directed against different determinants (epitopes), each monoclonal antibody is directed against a single determinant on the antigen. This type of antibodies is produced by the daughter cells of a single antibody-producing hybridoma. A monoclonal antibody typically displays a single binding affinity for any epitope with which it immunoreacts.

The modifier “monoclonal” indicates the character of the antibody as being obtained from a substantially homogeneous population of antibodies, and is not to be construed as requiring production of the antibody by any particular method. Monoclonal antibodies recognize only one type of antigen. The monoclonal antibodies herein include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies. The preparation of antibodies, whether monoclonal or polyclonal, is know in the art. Techniques for the production of antibodies are well known in the art and described, e.g. in Harlow and Lane “Antibodies, A Laboratory Manual”, Cold Spring Harbor Laboratory Press, 1988 and Harlow and Lane “Using Antibodies: A Laboratory Manual” Cold Spring Harbor Laboratory Press, 1999.

A monoclonal antibody may contain an antibody molecule having a plurality of antibody combining sites, each immunospecific for a different epitope, e.g., a bispecific monoclonal antibody. Monoclonal antibodies may be obtained by methods known to those skilled in the art. Kohler and Milstein (1975), Nature, 256:495-497; U.S. Pat. No. 4,376,110; Ausubel et al. (1987, 1992), eds., Current Protocols in Molecular Biology, Greene Publishing Assoc. and Wiley Interscience, N.Y.; Harlow and Lane (1988), Antibodies: A Laboratory Manual, Cold Spring Harbor Laboratory; Colligan et al. (1992, 1993), eds., Current Protocols in Immunology, Greene Publishing Assoc. and Wiley Interscience, N.Y.; Iyer et al., Ind. J. Med. Res., (2000), 123:561-564.

As used herein, an “antigen” is an entity which induces or evokes an immune response in an organism. An immune response is characterized by the reaction of the cells, tissues and/or organs of an organism to the presence of a foreign entity. Such an immune response typically leads to the production by the organism of one or more antibodies against the foreign entity, e.g., antigen or a portion of the antigen.

As used herein, the term “detect” refers to identification of the presence, absence or the amount of the object to be detected.

As used herein, the term “immunoassay” is an assay that uses an antibody to specifically bind an antigen (e.g. a marker). The immunoassay is characterized by the use of specific binding properties of a particular antibody to isolate, target, and/or quantify an antigen. Immunoassays may include, but are not limited to, a radioimmunoassay (RIA), enzyme immunoassay (EIA), Enzyme linked immunsorbent assay (ELISA), immunohistochemistry and western blotting.

The phrase “specifically (or selectively) binds” to an antibody or “specifically (or selectively) immunoreactive with” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to marker “X” from specific species such as rat, mouse, or human can be selected to obtain only those polyclonal antibodies that are specifically immunoreactive with marker “X” and not with other proteins, except for polymorphic variants and alleles of marker “X”. This selection may be achieved by subtracting out antibodies that cross-react with marker “X” molecules from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

As used herein, the term “marker” or “biomarker” in the context of the present invention refers to a polypeptide (of a particular apparent molecular weight) or nucleic acid, which is differentially present in a sample taken from a subject who is affiliated with a disease as compared to a comparable sample taken from a control subject (e.g. a subject with a negative diagnosis or normal and healthy subject). The term “biomarker” is used interchangeably with the term “marker.” The biomarkers are identified by, for example, molecular mass in Daltons, and include the masses centered around the identified molecular masses for each marker, affinity binding, nucleic acid detection, etc. The term “measuring” means methods which include detecting the presence or absence of marker(s) in the sample, quantifying the amount of marker(s) in the sample, and/or qualifying the type of biomarker. Measuring can be accomplished by methods known in the art and those further described herein, including but not limited to immunoassay. Any suitable methods can be used to detect and measure one or more of the markers described herein. These methods include, without limitation, mass spectrometry (e.g., laser desorption/ionization mass spectrometry), fluorescence (e.g. sandwich immunoassay), surface plasmon resonance, ellipsometry and atomic force microscopy.

As used herein, the terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

A “protein” means a polymer of amino acid residues linked together by peptide bonds. The term, as used herein, refers to proteins, polypeptides, and peptides of any size, structure, or function. Typically, however, a protein will be at least 50 amino acids long. In some instances the protein encoded is smaller than about 50 amino acids. In this case, the polypeptide is termed a peptide. If the protein is a short peptide, it will be at least about 10 amino acid residues long. A protein may be naturally occurring, recombinant, or synthetic, or any combination of these. A protein may also comprise a fragment of a naturally occurring protein or peptide. A protein may be a single molecule or may be a multi-molecular complex. The term protein may also apply to amino acid polymers in which one or more amino acid residues are an artificial chemical analogue of a corresponding naturally occurring amino acid.

The term “protein expression” refers to the process by which a nucleic acid sequence undergoes translation such that detectable levels of the amino acid sequence or protein are expressed.

A “fragment of a protein” as used herein, refers to a protein that is a portion of another protein. For example, fragments of proteins may comprise polypeptides obtained by digesting full-length protein isolated from cultured cells. In one embodiment, a protein fragment comprises at least about six amino acids. In another embodiment, the fragment comprises at least about ten amino acids. In yet another embodiment, the protein fragment comprises at least about sixteen amino acids.

The terms “amino acid” and “amino acids” refer to all naturally occurring L-alpha-amino acids. The amino acids are identified by either the one-letter or three-letter designations as follows: aspartic acid (Asp:D), isoleucine (Ile:I), threonine (Thr:T), leucine (Leu:L), serine (Ser:S), tyrosine (Tyr:Y), glutamic acid (Glu:E), phenylalanine (Phe:F), proline (Pro:P), histidine (His:H), glycine (Gly:G), lysine (Lys:K), alanine (Ala:A), arginine (Arg:R), cysteine (Cys:C), tryptophan (Trp:W), valine (Val:V), glutamine (Gln:Q) methionine (Met:M), asparagines (Asn:N), where the amino acid is listed first followed parenthetically by the three and one letter codes, respectively.

The term “amino acid sequence variant” refers to molecules with some differences in their amino acid sequences as compared to a native sequence. The amino acid sequence variants may possess substitutions, deletions, and/or insertions at certain positions within the amino acid sequence. Ordinarily, variants will possess at least about 70% homology to a native sequence, and preferably, they will be at least about 80%, more preferably at least about 90% homologous to a native sequence.

As used herein, the term “derivative” is used synonymously with the term “variant” and refers to a molecule that has been modified or changed in any way relative to a reference molecule or starting molecule. A “variant” includes, but is not limited to a mutated variant of a protein (substitutional, insertional, deletion and covalent variant). “Substitutional variants” when referring to proteins are those that have at least one amino acid residue in a native or starting sequence removed and a different amino acid inserted in its place at the same position. The substitutions may be single, where only one amino acid in the molecule has been substituted, or they may be multiple, where two or more amino acids have been substituted in the same molecule.

As used herein, “Insertional variants” when referring to proteins are those with one or more amino acids inserted immediately adjacent to an amino acid at a particular position in a native or starting sequence. “Immediately adjacent” to an amino acid means connected to either the alpha-carboxy or alpha-amino functional group of the amino acid. “Deletional variants,” when referring to proteins, are those with one or more amino acids in the native or starting amino acid sequence removed. Ordinarily, deletional variants will have one or more amino acids deleted in a particular region of the molecule.

A “mass spectrometer” refers to a gas phase ion spectrometer that measures a parameter that can be translated into mass-to-charge ratios of gas phase ions. Mass spectrometers generally include an ion source and a mass analyzer. Examples of mass spectrometers are time-of-flight (TOF), magnetic sector, quadnipole filter, ion trap, ion cyclotron resonance, electrostatic sector analyzer and hybrids of these. “Mass spectrometry” refers to the use of a mass spectrometer to detect gas phase ions. A “laser desorption mass spectrometer” refers to a mass spectrometer that uses laser energy as a means to desorb, volatilize, and ionize an analyte. A “tandem mass spectrometer” refers to any mass spectrometer that is capable of performing two successive stages of m/z-based discrimination or measurement of ions, including ions in an ion mixture. The phrase includes mass spectrometers having two mass analyzers that are capable of performing two successive stages of mlz-based discrimination or measurement of ions tandem-in-space. The phrase further includes mass spectrometers having a single mass analyzer that is capable of performing two successive stages of m/z-based discrimination or measurement of ions tandem-in-time. The phrase thus explicitly includes Qq-TOF mass spectrometers, ion trap mass spectrometers, ion trap-TOF mass spectrometers, TOF-TOF mass spectrometers, Fourier transform ion cyclotron resonance mass spectrometers, electrostatic sector-magnetic sector mass spectrometers, and combinations thereof.

As used herein, the term “ionization” refers to the process by which analytes in a sample are ionized. Such analytes may become charged molecules used for further analysis. For example, sample ionization may be performed by electrospray ionization (ESI), atmospheric pressure chemical ionization (APCI), photoionization, electron ionization, fast atom bombardment (FAB)/liquid secondary ionization (LSIMS), matrix assisted laser desorption ionization (MALDI), field ionization, field desorption, thermospray/plasmaspray ionization, and particle beam ionization. The skilled artisan will understand that the choice of ionization method can be determined based on the analyte to be measured, type of sample, the type of detector, the choice of positive versus negative mode, etc.

A “mass analyzer” refers to the component of the mass spectrometer that takes ionized masses and separates them based on charge to mass ratios and outputs them to the detector where they are detected and later converted to a digital output. Suitable mass analyzers for determining mass-to-charge ratios include quadrupole mass analyzer, time-of-flight (TOF) mass analyzer, magnetic or electrostatic sector mass analyzer and ion trap (e.g. ion cyclotron resonance) mass analyzer.

As used herein, the term “selected ion monitoring” or “SIM” refers to a mass spectrometry scanning mode in which a limited mass-to-charge ratio range is transmitted and/or detected by the mass spectrometer, as opposed to the full spectrum range, which results in significantly increased sensitivity.

As used herein, the term “single/selected reaction monitoring” or “SRM” refers to a scanning mode to select and analyze a specific analyte (e.g. a peptide or a small molecule) utilizing a triple quadrupole mass spectrometer. In SRM analysis, the specificity depends on multiple mass analyzers (mass filters.) The first quadrupole is to select the desired parent ion. The third quadrupole is to monitor the (one or more) fragment ion(s). The fragment ion(s) is generated through collisional induced dissociation in the second quadrupole. Therefore, SRM is a highly specific detection/monitoring method with low background interference. When multiple parent ions are monitored in a single MS run, this type of analysis is known as “multiple reaction monitoring (MRM)”. Using MRM analysis, multiple proteins and multiple regions (signature peptides) of a protein can be monitored in single mass spectrometry run.

As used herein, the “mass spectrometry profile” refers to one or more proteins or a group of peptides from a sample isolated from a subject wherein the presence and the concentration of proteins or peptides, taken individually or together, is indicative/predictive of a disease.

The term “predicting” means a statement or claim that a particular event will, or is likely to, occur in the future.

The term “prognosing” means a statement or claim that a particular biological event will, or is likely to, occur in the future.

The invention is further illustrated by the following non-limiting examples.

### EXAMPLES

### Example 1

### Selection of Proteotypic Peptides and SRM Method Generation

Full length recombinant A2M protein was purchased from Origene Technologies Inc (Rockville, Md.). After separation on SDS-PAGE by molecular weight, the protein band was excised and reduced/alkylated before trypsin digestion. An Agilent 6520 Q-TOF was used to create tryptic peptide profiles for each in-gel digested recombinant protein (Agilent Technologies, Santa Clara, Calif.). The tryptic peptides were then identified using Spectrum Mill and MASCOT, two search engines developed by Agilent and Matrix Science (Boston, Mass.), respectively. Peptides that were identified by the search engines and with high scores (score >6 for Spectrum Mill, score >10 for MASCOT) were then subjected to the Peptide Optimizer software (Agilent Technologies, Santa Clara, Calif.) to optimize the collision energy for each transition (the mass/charge value of an intact peptide vs. the mass/charge value of fragmented peptides). In the transition list, only singly charged y ions, which were generated by breaking down the amide bonds of an intact peptide in the collision cells of the Mass spectrometry, were considered. In addition, the mass/charge value of each y ion is larger than the mass/charge value of its corresponding intact peptide.

The final SRM method included 31 transitions from 9 A2M peptides with optimized collision energies. The QQQ was set to operate in a targeted fashion whereby only molecular ions corresponding to the most dominant charge state of +2, +3 or +4 of selected peptides were transmitted through Q1, and SRM transition candidates were monitored in Q3. Both Q1 and Q3 resolution were set to “unit”, and a default dwell time of 5 or 10 milliseconds was used.

### Example 2

### Immuno-Capture of A2M Protein Using MSIA Technology

The immuno-capture step was performed using an automated liquid handling or pipetting instrument. The anti-A2M antibody was purchased from R&D systems, Inc (Minneapolis, Minn.). The human plasma sample was purchased from Research Blood Components, LLC (Boston, Mass.). The antibody was diluted into PBS buffer with 0.1% tween to reach the final concentration of 0.1 mg/ml. The human plasma was diluted into the same PBS buffer by the dilution factor of 4. Protein A/protein G MSIA tips from Thermo Scientific Inc (Tempe, Ariz.) were used to generate affinity tips for capturing and concentrating the protein of interest from human plasma. Antibody solutions (100 IA) were flowed 500 times with repetitive aspirations and dispenses through the tips. The antibodies then bound to protein A/protein G that were non-covalently conjugated to the tips to generate antibody-based affinity tips. After eight complete washes with PBS, the human plasma sample (100 IA) was flowed 500 times through the affinity tips. A PBS buffer followed by water was used to aggressively wash away unbound compounds such as proteins, salts and other molecules in the sample. The A2M protein retained on the tips was eluted to 50 μl of 30% acetonitrile (ACN) (0.5% formic acid) and followed by another elution step using 50 μl of 50% ACN (0.5% formic acid) to generate samples concentrated with proteins captured by the affinity tips. The concentrated samples were pooled together and dried to less than 5 μl using an Eppendorf speed vacuum concentrator (Eppendorf, Hauppauge, N.Y.). The resulting solution was combined with 25 μl of 0.2 M ammonium bicarbonate and 4 M urea buffer before preceding to reduction/alkylation steps followed by trypsin digestion to generate peptides for mass spectrometry analysis.

### Example 3

### LC-QQQ-SRM Mass Spectrometry

Liquid chromatography was performed using a 1200 Series LC system interfaced to a 6410 (Nuclea Biotechnologies, Pittsfield, Mass.)) Triple Quadrupole (QQQ) LC/MS/MS (Agilent Technologies, Santa Clara, Calif.). Agilent MassHunter software (version B.03.01) was used for data acquisition and processing. The LC separation of peptides was carried out on a Zorbax 300SB-C18 5-μm column (Agilent Technologies, Santa Clara, Calif.).

For analysis of tryptic peptides, processed peptides were loaded onto the column using an Agilent 1260 autosampler. The gradient separation was performed by the capillary LC pump delivering a mixture of 99.9% water/0.1% formic acid (mobile phase A) and 99.9% acetonitrile/0.1% formic acid (mobile phase B) at 400 μL/min. Peptides were separated at a flow rate of 4004/min by a nanopump delivering a linear gradient of 2 to 38.8% mobile phase Bin 35 minutes followed by 38.8 to 95% mobile phase B in 2 minute.

The analyses were performed in the positive ionization mode with a capillary voltage set at 4000 V and an electron multiplier voltage (Delta EMV) (Agilent, Santa Clara, Calif.) at 350 V. The drying gas flow rate was 10 L nitrogen/min with an interface heater temperature of 350° C. The MS fragmentor voltage was fixed at 135 V. Selected reaction monitoring (SRM) transition dwell times were 5 ms, with both Q1 and Q3 set to “unit”.

### Example 4

### Selected MSIA-SRM Peptides of A2M Protein

Certain peptides and peptide signatures were identified which may be useful in the determination of the concentration or presence of A2M. The signature peptides identified through the MSIA-SRM assay are listed in Table 1. In the Table, the peptide No. represents the SRM peptide number, m/z represents the mass over charge of precursor ion, Sequence represents the peptide sequence of SRM and MS2 represents the mass over charge of the product ions.

TABLE 1
Sequence of MSIA-SRM peptides and MS data for A2M

| Peptide | | | | | | | SEQ |
|---|---|---|---|---|---|---|---|
| No | Sequence | MS1 | MS2 | MS2 | MS2 | MS2 | ID NO |
| 1 | SLPTDLEAENDVLHCV | 825.7 | 1138.1 | 1064.5 | 956.5 | 835.4 | 3 |
| | AFAVPK | | | | | | |
| 2 | AIGYLNTGYQR | 628.3 | 1071.5 | 851.4 | 738.4 | | 4 |
| 3 | LPPNVVEESAR | 605.8 | 1000.5 | 903.5 | 789.4 | 690.3 | 5 |
| 4 | DTVIKPLLVEPEGLEK | 594.0 | 801.4 | 782.5 | 732.9 | 672.4 | 6 |
| 5 | QGIPFFGQVR | 574.8 | 850.5 | 753.4 | 606.3 | | 7 |
| 6 | FEVQVTVPK | 523.8 | 770.5 | 671.4 | 543.4 | | 8 |
| 7 | LVHVEEPHTETVR | 515.8 | 667.3 | 605.3 | 598.8 | | 9 |
| 8 | FQVDNNNR | 503.7 | 731.3 | 632.3 | 517.2 | | 10 |
| 9 | YGAATFTR | 443.7 | 723.4 | 666.4 | 595.3 | 524.3 | 11 |

### Examples 5

### MSIA-SRM Assay for FASN

Application of MSIA to FASN utilized an anti-FASN monoclonal antibody generated by Nuclea Biotechnologies (Pittsfield, Mass.). The antibodies were diluted in solution and directly conjugated to the MSIA tips (Thermo Scientific, Waltham, Mass.) to generate affinity tips used in the assay with “covalent” bonding of the antibodies with the resin in the tip. 200 μl of HOP-62 cell lysate (American Tissue Culture Collection (ATCC), Manassas, Va.) was flowed 500 times with repetitive aspirations and dispenses through the anti-FASN antibody-based affinity tips. A PBS buffer followed by water was used to aggressively wash away unbound compounds such as proteins, salts and other molecules. Captured FASN was eluted from the tips with 50 μl of 30% Acetonitrile (ACN) (0.5% formic acid) to generate FASN concentrated samples. The concentrated samples were pooled together and dried using an Eppendorf speed vacuum concentrator (Hauppauge, N.Y.). The resulting solution was combined with 50 μl of buffer comprising 4 M Urea, 300 mM Tris-HCL and 2.5% N-propanol (pH 8.5) and subjected to reduction alkylation steps followed by trypsin digestion to generate peptides for the mass spec analysis. SRM assays were performed as described in Example 3.

Certain peptides and peptide signatures were identified which may be useful in the determination of the concentration or presence of FASN. The signature peptides identified through the MSIA-SRM assay are listed in Table 2. The peptide No. represents the MRM peptide number, MS1 represents the mass over charge of precursor ion, Sequence represents the peptide sequence of MRM, and MS2 represents the mass over charge of the product ions.

TABLE 2
Sequence of MSIA-SRM peptides and MS data for FASN

| Peptide | | | | | | | SEQ |
|---|---|---|---|---|---|---|---|
| No. | Sequence | MS1 | MS2 | MS2 | MS2 | MS2 | ID NO |
| 1 | AGLYGLPR | 423.74 | 442.28 | 605.34 | 718.42 | 775.44 | 12 |
| 2 | LQVVDQPLPVR | 632.38 | 581.38 | 824.46 | 923.53 | 1022.60 | 13 |
| 3 | LLEQGLR | 414.75 | 473.28 | 602.32 | 715.41 | | 14 |
| 4 | GYAVLGGER | 461.24 | 418.20 | 531.29 | 630.36 | 701.39 | 15 |
| 5 | GTPLISPLIK | 519.83 | 557.36 | 670.45 | 783.53 | 440.80 | 16 |
| 6 | TGTVSLEVR | 481.27 | 516.31 | 603.34 | 702.41 | 803.46 | 17 |
| 7 | HGLYLPTR | 478.77 | 486.30 | 649.37 | 762.45 | 819.47 | 18 |
| 8 | LYTLQDK | 440.74 | 503.28 | 604.33 | 767.39 | | 19 |
| 9 | AQVADVVVSR | 522.30 | 460.29 | 674.38 | 745.42 | 844.49 | 20 |
| 10 | GLVQALQTK | 479.29 | 489.30 | 560.34 | 688.40 | 787.47 | 21 |
| 11 | DPSQQELPR | 535.27 | 514.30 | 642.36 | 770.42 | 857.45 | 22 |
| 12 | DGAWGAFR | 440.21 | 450.24 | 636.32 | 707.36 | 764.38 | 23 |
| 13 | LSPDAIPGK | 449.26 | 485.31 | 600.33 | 697.39 | 784.42 | 24 |
| 14 | VFTTVGSAEK | 519.78 | 491.24 | 590.31 | 691.36 | 792.41 | 25 |
| 15 | YSGTLNLDR | 519.76 | 517.27 | 630.36 | 788.42 | 875.46 | 26 |
| 16 | ELNLVLSVR | 521.82 | 474.30 | 573.37 | 686.46 | 800.50 | 27 |
| 17 | LQELSSK | 402.73 | 434.26 | 563.30 | 691.36 | | 28 |
| 18 | QVQPEGPYR | 537.27 | 492.26 | 621.30 | 718.35 | 846.41 | 29 |
| 19 | VLEALLPLK | 498.33 | 470.33 | 583.42 | 654.45 | 783.50 | 30 |
| 20 | VAAAVDLIIK | 506.82 | 700.46 | 771.50 | 842.53 | 913.57 | 31 |
| 21 | SHQGLDR | 406.70 | 403.23 | 460.25 | 588.31 | 725.37 | 32 |
| 22 | QELSFAAR | 461.24 | 464.26 | 551.29 | 664.38 | 793.42 | 33 |
| 23 | AAEQYTPK | 454.23 | 508.28 | 636.33 | 765.38 | 836.41 | 34 |

### Example 6

### Construction of Calibration Curves of MSIA Assay

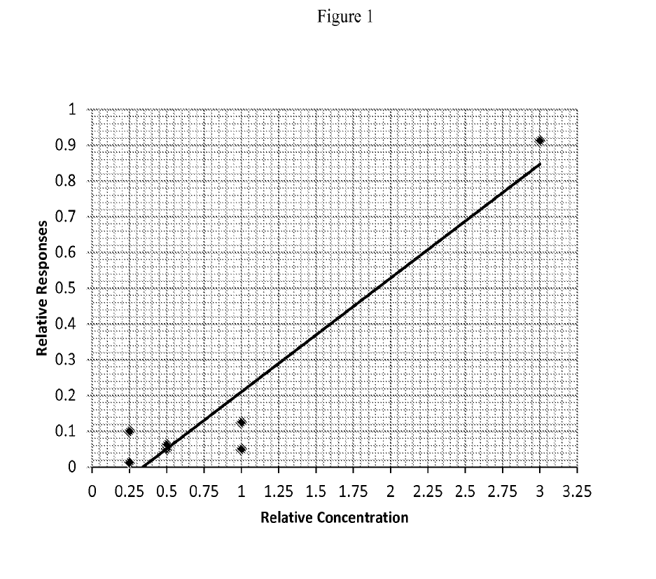

Calibration curves of this assay were created using recombinant proteins A2M and FASN. Recombinant proteins, A2M or FASN were purchased from Origene (Rockville, Md.). The concentrations of A2M protein were 1.25, 2.5, 5 and 15 ug/mL with each data point representing the average from duplicate analysis. A heavy labeled A2M from Origene with a concentration of 5 μg/mL was used as an internal standard. The linear calibration curve for each detected peptide was generated by plotting the ratio of peak area (sum of the peak area from each non-labeled peptide transition to sum of the peak area from each labeled peptide transition) vs the ratio of the contraction (non-labeled protein to labeled protein). An example of the calibration curve from A2M peptide AIGYLNTGYQR (SEQ ID NO. 4) with R2 equal to 0.91 is shown in FIG. 1. The concentrations of recombinant FASN protein were 0, 1.25, 2.5, 5, 10, 20, and 40 ng/mL with each data point representing the average of triplicate values. The curve was generated by plotting the peak area (sum of the peak area from peptide transition) vs. the concentration value. An example of the calibration curve from FASN peptide GYAVLGGER (SEQ ID NO. 15) with R2 equal to 0.98 is shown in FIG. 2.

### EQUIVALENTS AND SCOPE

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

1-33. (canceled)

34. A method for quantifying fatty acid synthase (FASN) protein in a biological sample comprising:
(a) obtaining a biological sample from a subject;
(b) concentrating FASN protein from the sample, wherein the step of concentrating FASN comprises capturing FASN in the sample using an antibody, dissociating the captured FASN from the antibody, and proteolytically digesting the dissociated FASN protein;
(c) generating a mass spectrometric profile of the digested FASN fragment peptides by mass spectrometry;
(d) comparing the mass spectrometric profile of the FASN fragment peptides from (c) to a standard calibration curve, wherein said standard calibration curve is created using at least one calibration standard peptide for FASN selected from the group consisting of SEQ ID NO. 12, SEQ ID NO. 13, SEQ ID NO. 14, SEQ ID NO. 15, SEQ ID NO.16, SEQ ID NO. 17, SEQ ID NO. 18, SEQ ID NO. 19, SEQ ID NO. 20, SEQ ID NO. 21, SEQ ID NO. 22, SEQ ID NO. 23, SEQ ID NO. 24, SEQ ID NO. 25, SEQ ID NO. 26, SEQ ID NO. 27, SEQ ID NO. 28, SEQ ID NO. 29, SEQ ID NO. 30, SEQ ID NO. 31, SEQ ID NO. 32, SEQ ID NO. 33, SEQ ID NO. 34 and any combinations thereof; and
(e) quantifying the FASN protein in the sample from the subject based on the standard calibration curve,
wherein the quantitation is a relative quantitation or an absolute concentration.

35. The method of claim 34, wherein FASN comprises an amino acid sequence comprising SEQ ID NO.1.

36. The method of claim 35, wherein the antibody is an anti-FASN antibody.

37. The method of claim 34 further comprising adding into the sample a known concentration of one or more peptides selected from the group consisting of SEQ ID NO. 12, SEQ ID NO. 13, SEQ ID NO. 14, SEQ ID NO. 15, SEQ ID NO.16, SEQ ID NO. 17, SEQ ID NO. 18, SEQ ID NO. 19, SEQ ID NO. 20, SEQ ID NO. 21, SEQ ID NO. 22, SEQ ID NO. 23, SEQ ID NO. 24, SEQ ID NO. 25, SEQ ID NO. 26, SEQ ID NO. 27, SEQ ID NO. 28, SEQ ID NO. 29, SEQ ID NO. 30, SEQ ID NO. 31, SEQ ID NO. 32, SEQ ID NO. 33, SEQ ID NO. 34, and/or any combinations thereof.

38. A synthetic isolated peptide 6 to 22 amino acids in length having at least 5 contiguous amino acids of a peptide selected from the group consisting of SEQ ID NO. 3, SEQ ID NO. 4, SEQ ID NO. 5, SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, SEQ ID NO. 14, SEQ ID NO. 15, SEQ ID NO.16, SEQ ID NO. 17, SEQ ID NO. 18, SEQ ID NO. 19, SEQ ID NO. 20, SEQ ID NO. 21, SEQ ID NO. 22, SEQ ID NO. 23, SEQ ID NO. 24, SEQ ID NO. 25, SEQ ID NO. 26, SEQ ID NO. 27, SEQ ID NO. 28, SEQ ID NO. 29, SEQ ID NO. 30, SEQ ID NO. 31, SEQ ID NO. 32, SEQ ID NO. 33 and SEQ ID NO. 34.

39. The synthetic peptide of claim 38 where the peptide is from 6-13 amino acids in length.

40. The synthetic peptide of claim 38 where the peptide is from 8-22 amino acids in length.

41. The synthetic peptide of claim 38 further comprising at least one detectable label.

42. The synthetic peptide of claim 38, wherein the at least one detectable label is selected from the group consisting of a fluorescent label, nitrogen-15, carbon-13, hydrogen-2, sulfur-34, oxygen-18, oxygen-17 and deuterium.

43. A kit used to quantify the concentration of the protein biomarker FASN in a sample comprising two or more standard peptides selected from the group consisting of SEQ ID NO. 12, SEQ ID NO. 13, SEQ ID NO. 14, SEQ ID NO. 15, SEQ ID NO.16, SEQ ID NO. 17, SEQ ID NO. 18, SEQ ID NO. 19, SEQ ID NO. 20, SEQ ID NO. 21, SEQ ID NO. 22, SEQ ID NO. 23, SEQ ID NO. 24, SEQ ID NO. 25, SEQ ID NO. 26, SEQ ID NO. 27, SEQ ID NO. 28, SEQ ID NO. 29, SEQ ID NO. 30, SEQ ID NO. 31, SEQ ID NO. 32, SEQ ID NO. 33, SEQ ID NO. 34 and any combinations thereof.

44. The kit of claim 43 further comprising an anti-FASN antibody.


SEQUENCE LISTING (34 sequences)

SEQ ID NO: 1 - DNA, 8481 bp, Homo sapiens
  CDS (118)..(7653)
gagagacggcagcggccccggcctccctctccgccgcgcttcagcctcccgctccgccgcgctccagcctcgctctccgccgcccgcaccgccgcccgcgccctcaccagagcagccatggaggaggtggtgattgccggcatgtccgggaagctgccagagtcggagaacttgcaggagttctgggacaacctcatcggcggtgtggacatggtcacggacgatgaccgtcgctggaaggcggggctctacggcctgccccggcggtccggcaagctgaaggacctgtctaggtttgatgcctccttcttcggagtccaccccaagcaggcacacacgatggaccctcagctgcggctgctgctggaagtcacctatgaagccatcgtggacggaggcatcaacccagattcactccgaggaacacacactggcgtctgggtgggcgtgagcggctctgagacctcggaggccctgagccgagaccccgagacactcgtgggctacagcatggtgggctgccagcgagcgatgatggccaaccggctctccttcttcttcgacttcagagggcccagcatcgcactggacacagcctgctcctccagcctgatggccctgcagaacgcctaccaggccatccacagcgggcagtgccctgccgccatcgtggggggcatcaatgtcctgctgaagcccaacacctccgtgcagttcttgaggctggggatgctcagccccgagggcacctgcaaggccttcgacacagcggggaatgggtactgccgctcggagggtgtggtggccgtcctgctgaccaagaagtccctggcccggcgggtgtacgccaccatcctgaacgccggcaccaatacagatggcttcaaggagcaaggcgtgaccttcccctcaggggatatccaggagcagctcatccgctcgttgtaccagtcggccggagtggcccctgagtcatttgaatacatcgaagcccacggcacaggcaccaaggtgggcgacccccaggagctgaatggcatcacccgagccctgtgcgccacccgccaggagccgctgctcatcggctccaccaagtccaacatggggcacccggagccagcctcggggctggcagccctggccaaggtgctgctgtccctggagcacgggctctgggcccccaacctgcacttccatagccccaaccctgagatcccagcgctgttggatgggcggctgcaggtggtggaccagcccctgcccgtccgtggcggcaacgtgggcatcaactcctttggcttcgggggctccaacgtgcacatcatcctgaggcccaacacgcagccgccccccgcacccgccccacatgccaccctgccccgtctgctgcgggccagcggacgcacccctgaggccgtgcagaagctgctggagcagggcctccggcacagccaggacctggctttcctgagcatgctgaacgacatcgcggctgtccccgccaccgccatgcccttccgtggctacgctgtgctgggtggtgagcgcggtggcccagaggtgcagcaggtgcccgctggcgagcgcccgctctggttcatctgctctgggatgggcacacagtggcgcgggatggggctgagcctcatgcgcctggaccgcttccgagattccatcctacgctccgatgaggctgtgaagccattcggcctgaaggtgtcacagctgctgctgagcacagacgagagcacctttgatgacatcgtccattcgtttgtgagcctgactgccatccagataggcctcatagacctgctgagctgcatggggctgaggccagatggcatcgtcggccactccctgggggaggtggcctgtggctacgccgacggctgcctgtcccaggaggaggccgtcctcgctgcctactggaggggacagtgcatcaaagaagcccatctcccgccgggcgccatggcagccgtgggcttgtcctgggaggagtgtaaacagcgctgccccccgggcgtggtgcccgcctgccacaactccaaggacacagtcaccatctcgggacctcaggccccggtgtttgagttcgtggagcagctgaggaaggagggtgtgtttgccaaggaggtgcggaccggcggtatggccttccactcctacttcatggaggccatcgcacccccactgctgcaggagctcaagaaggtgatccgggagccgaagccacgttcagcccgctggctcagcacctctatccccgaggcccagtggcacagcagcctggcacgcacgtcctccgccgagtacaatgtcaacaacctggtgagccctgtgctgttccaggaggccctgtggcacgtgcctgagcacgcggtggtgctggagatcgcgccccacgccctgctgcaggctgtcctgaagcgtggcctgaagccgagctgcaccatcatccccctgatgaagaaggatcacagggacaacctggagttcttcctggccggcatcggcaggctgcacctctcaggcatcgacgccaaccccaatgccttgttcccacctgtggagttcccagctccccgaggaactcccctcatctccccactcatcaagtgggaccacagcctggcctgggacgtgccggccgccgaggacttccccaacggttcaggttccccctcagccgccatctacaacatcgacaccagctccgagtctcctgaccactacctggtggaccacaccctcgacggtcgcgtcctcttccccgccactggctacctgagcatagtgtggaagacgctggcccgcgccctgggcctgggcgtcgagcagctgcctgtggtgtttgaggatgtggtgctgcaccaggccaccatcctgcccaagactgggacagtgtccctggaggtacggctcctggaggcctcccgtgccttcgaggtgtcagagaacggcaacctggtagtgagtgggaaggtgtaccagtgggatgaccctgaccccaggctcttcgaccacccggaaagccccacccccaaccccacggagcccctcttcctggcccaggctgaagtttacaaggagctgcgtctgcgtggctacgactacggccctcatttccagggcatcctggaggccagcctggaaggtgactcggggaggctgctgtggaaggataactgggtgagcttcatggacaccatgctgcagatgtccatcctgggctcggccaagcacggcctgtacctgcccacccgtgtcaccgccatccacatcgaccctgccacccacaggcagaagctgtacacactgcaggacaaggcccaagtggctgacgtggtggtgagcaggtggctgagggtcacagtggccggaggcgtccacatctccgggctccacactgagtcggccccgcggcggcagcaggagcagcaggtgcccatcctggagaagttttgcttcactccccacacggaggaggggtgcctgtctgagcgcgctgccctgcaggaggagctgcaactgtgcaaggggctggtgcaggcactgcagaccaaggtgacccagcaggggctgaagatggtggtgcccggactggatggggcccagatcccccgggacccctcacagcaggaactgccccggctgttgtcggctgcctgcaggcttcagctcaacgggaacctgcagctggagctggcgcaggtgctggcccaggagaggcccaagctgccagaggaccctctgctcagcggcctcctggactccccggcactcaaggcctgcctggacactgccgtggagaacatgcccagcctgaagatgaaggtggtggaggtgctggctggccacggtcacctgtattcccgcatcccaggcctgctcagcccccatcccctgctgcagctgagctacacggccaccgaccgccacccccaggccctggaggctgcccaggccgagctgcagcagcacgacgttgcccagggccagtgggatcccgcagaccctgcccccagcgccctgggcagcgccgacctcctggtgtgcaactgtgctgtggctgccctcggggacccggcctcagctctcagcaacatggtggctgccctgagagaagggggctttctgctcctgcacacactgctccgggggcaccccctcggggacatcgtggccttcctcacctccactgagccgcagtatggccagggcatcctgagccaggacgcgtgggagagcctcttctccagggtgtcgctgcgcctggtgggcctgaagaagtccttctacggctccacgctcttcctgtgccgccggcccaccccgcaggacagccccatcttcctgccggtggacgataccagcttccgctgggtggagtctctgaagggcatcctggctgacgaagactcttcccggcctgtgtggctgaaggccatcaactgtgccacctcgggcgtggtgggcttggtgaactgtctccgccgagagcccggcgggaaccgcctccggtgtgtgctgctctccaacctcagcagcacctcccacgtcccggaggtggacccgggctccgcagaactgcagaaggtgttgcagggagacctggtgatgaacgtctaccgcgacggggcctggggggctttccgccacttcctgctggaggaggacaagcctgaggagccgacggcacatgcctttgtgagcaccctcacccggggggacctgtcctccatccgctgggtctgctcctcgctgcgccatgcccagcccacctgccctggcgcccagctctgcacggtctactacgcctccctcaacttccgcgacatcatgctggccactggcaagctgtcccctgatgccatcccagggaagtggacctcccaggacagcctgctaggtatggagttctcgggccgagacgccagcggcaagcgtgtgatgggactggtgcctgccaagggcctggccacctctgtcctgctgtcaccggacttcctctgggatgtgccttccaactggacgctggaggaggcggcctcggtgcctgtcgtctacagcacggcctactacgcgctggtggtgcgtgggcgggtgcgccccggggagacgctgctcatccactcgggctcgggcggcgtgggccaggccgccatcgccatcgccctcagtctgggctgccgcgtcttcaccaccgtggggtcggctgagaagcgggcgtacctccaggccaggttcccccagctcgacagcaccagcttcgccaactcccgggacacatccttcgagcagcatgtgctgtggcacacgggcgggaagggcgttgacctggtcttgaactccttggcggaagagaagctgcaggccagcgtgaggtgcttggctacgcacggtcgcttcctggaaattggcaaattcgacctttctcagaaccacccgctcggcatggctatcttcctgaagaacgtgacattccacggggtcctactggatgcgttcttcaacgagagcagtgctgactggcgggaggtgtgggcgcttgtgcaggccggcatccgggatggggtggtacggcccctcaagtgcacggtgttccatggggcccaggtggaggacgccttccgctacatggcccaagggaagcacattggcaaagtcgtcgtgcaggtgcttgcggaggagccggaggcagtgctgaagggggccaaacccaagctgatgtcggccatctccaagaccttctgcccggcccacaagagctacatcatcgctggtggtctgggtggcttcggcctggagttggcgcagtggctgatacagcgtggggtgcagaagctcgtgttgacttctcgctccgggatccggacaggctaccaggccaagcaggtccgccggtggaggcgccagggcgtacaggtgcaggtgtccaccagcaacatcagctcactggagggggcccggggcctcattgccgaggcggcgcagcttgggcccgtgggcggcgtcttcaacctggccgtggtcttgagagatggcttgctggagaaccagaccccagagttcttccaggacgtctgcaagcccaagtacagcggcaccctgaacctggacagggtgacccgagaggcgtgccctgagctggactactttgtggtcttctcctctgtgagctgcgggcgtggcaatgcgggacagagcaactacggctttgccaattccgccatggagcgtatctgtgagaaacgccggcacgaaggcctcccaggcctggccgtgcagtggggcgccatcggcgacgtgggcattttggtggagacgatgagcaccaacgacacgatcgtcagtggcacgctgccccagcgcatggcgtcctgcctggaggtgctggacctcttcctgaaccagccccacatggtcctgagcagctttgtgctggctgagaaggctgcggcctatagggacagggacagccagcgggacctggtggaggccgtggcacacatcctgggcatccgcgacttggctgctgtcaacctggacagctcactggcggacctgggcctggactcgctcatgagcgtggaggtgcgccagacgctggagcgtgagctcaacctggtgctgtccgtgcgcgaggtgcggcaactcacgctccggaaactgcaggagctgtcctcaaaggcggatgaggccagcgagctggcatgccccacgcccaaggaggatggtctggcccagcagcagactcagctgaacctgcgctccctgctggtgaacccggagggccccaccctgatgcggctcaactccgtgcagagctcggagcggcccctgttcctggtgcacccaatcgagggctccaccaccgtgttccacagcctggcctcccggctcagcatccccacctatggcctgcagtgcacccgagctgcgccccttgacagcatccacagcctggctgcctactacatcgactgcatcaggcaggtgcagcccgagggcccctaccgcgtggccggctactcctacggggcctgcgtggcctttgaaatgtgctcccagctgcaggcccagcagagcccagcccccacccacaacagcctcttcctgttcgacggctcgcccacctacgtactggcctacacccagagctaccgggcaaagctgaccccaggctgtgaggctgaggctgagacggaggccatatgcttcttcgtgcagcagttcacggacatggagcacaacagggtgctggaggcgctgctgccgctgaagggcctagaggagcgtgtggcagccgccgtggacctgatcatcaagagccaccagggcctggaccgccaggagctgagctttgcggcccggtccttctactacaagctgcgtgccgctgagcagtacacacccaaggccaagtaccatggcaacgtgatgctactgcgcgccaagacgggtggcgcctacggcgaggacctgggcgcggactacaacctctcccaggtatgcgacgggaaagtatccgtccacgtcatcgagggtgaccaccgcacgctgctggagggcagcggcctggagtccatcatcagcatcatccacagctccctggctgagccacgcgtgagcgtgcgggagggctaggcccgtgcccccgcctgccaccggaggtcactccaccatccccaccccaccccaccccacccccgccatgcaacgggattgaagggtcctgccggtgggaccctgtccggcccagtgccactgccccccgaggctgctagatgtaggtgttaggcatgtcccacccacccgccgcctcccacggcacctcggggacaccagagctgccgacttggagactcctggtctgtgaagagccggtggtgcccgtgcccgcaggaactgggctgggcctcgtgcgcccgtggggtctgcgcttggtctttctgtgcttggatttgcatatttattgcattgctggtagagacccccaggcctgtccaccctgccaagactcctcaggcagcgtgtgggtcccgcactctgcccccatttccccgatgtcccctgcgggcgcgggcagccacccaagcctgctggctgcggccccctctcggccaggcattggctcagcccgctgagtggggggtcgtgggccagtccccgaggagctgggcccctgcacaggcacacagggcccggccacacccagcggccccccgcacagccacccgtggggtgctgcccttatgcccggcgccgggcaccaactccatgtttggtgtttgtctgtgtttgtttttcaagaaatgattcaaattgctgcttggattttgaaatttactgtaactgtcagtgtacacgtctggaccccgtttcatttttacaccaatttggtaaaaatgctgctctcagcctcccacaattaaaccgcatgtgatctccaaaaaaaaaaaaaaaaaaaaaaaa

SEQ ID NO: 2 - DNA, 4678 bp, Homo sapiens
  CDS (114)..(4538)
gcacacagagcagcataaagcccagttgctttgggaagtgtttgggaccagatggattgtagggagtagggtacaatacagtctgttctcctccagctccttctttctgcaacatggggaagaacaaactccttcatccaagtctggttcttctcctcttggtcctcctgcccacagacgcctcagtctctggaaaaccgcagtatatggttctggtcccctccctgctccacactgagaccactgagaagggctgtgtccttctgagctacctgaatgagacagtgactgtaagtgcttccttggagtctgtcaggggaaacaggagcctcttcactgacctggaggcggagaatgacgtactccactgtgtcgccttcgctgtcccaaagtcttcatccaatgaggaggtaatgttcctcactgtccaagtgaaaggaccaacccaagaatttaagaagcggaccacagtgatggttaagaacgaggacagtctggtctttgtccagacagacaaatcaatctacaaaccagggcagacagtgaaatttcgtgttgtctccatggatgaaaactttcaccccctgaatgagttgattccactagtatacattcaggatcccaaaggaaatcgcatcgcacaatggcagagtttccagttagagggtggcctcaagcaattttcttttcccctctcatcagagcccttccagggctcctacaaggtggtggtacagaagaaatcaggtggaaggacagagcaccctttcaccgtggaggaatttgttcttcccaagtttgaagtacaagtaacagtgccaaagataatcaccatcttggaagaagagatgaatgtatcagtgtgtggcctatacacatatgggaagcctgtccctggacatgtgactgtgagcatttgcagaaagtatagtgacgcttccgactgccacggtgaagattcacaggctttctgtgagaaattcagtggacagctaaacagccatggctgcttctatcagcaagtaaaaaccaaggtcttccagctgaagaggaaggagtatgaaatgaaacttcacactgaggcccagatccaagaagaaggaacagtggtggaattgactggaaggcagtccagtgaaatcacaagaaccataaccaaactctcatttgtgaaagtggactcacactttcgacagggaattcccttctttgggcaggtgcgcctagtagatgggaaaggcgtccctataccaaataaagtcatattcatcagaggaaatgaagcaaactattactccaatgctaccacggatgagcatggccttgtacagttctctatcaacaccaccaatgttatgggtacctctcttactgttagggtcaattacaaggatcgtagtccctgttacggctaccagtgggtgtcagaagaacacgaagaggcacatcacactgcttatcttgtgttctccccaagcaagagctttgtccaccttgagcccatgtctcatgaactaccctgtggccatactcagacagtccaggcacattatattctgaatggaggcaccctgctggggctgaagaagctctccttctattatctgataatggcaaagggaggcattgtccgaactgggactcatggactgcttgtgaagcaggaagacatgaagggccatttttccatctcaatccctgtgaagtcagacattgctcctgtcgctcggttgctcatctatgctgttttacctaccggggacgtgattggggattctgcaaaatatgatgttgaaaattgtctggccaacaaggtggatttgagcttcagcccatcacaaagtctcccagcctcacacgcccacctgcgagtcacagcggctcctcagtccgtctgcgccctccgtgctgtggaccaaagcgtgctgctcatgaagcctgatgctgagctctcggcgtcctcggtttacaacctgctaccagaaaaggacctcactggcttccctgggcctttgaatgaccaggacgatgaagactgcatcaatcgtcataatgtctatattaatggaatcacatatactccagtatcaagtacaaatgaaaaggatatgtacagcttcctagaggacatgggcttaaaggcattcaccaactcaaagattcgtaaacccaaaatgtgtccacagcttcaacagtatgaaatgcatggacctgaaggtctacgtgtaggtttttatgagtcagatgtaatgggaagaggccatgcacgcctggtgcatgttgaagagcctcacacggagaccgtacgaaagtacttccctgagacatggatctgggatttggtggtggtaaactcagcaggtgtggctgaggtaggagtaacagtccctgacaccatcaccgagtggaaggcaggggccttctgcctgtctgaagatgctggacttggtatctcttccactgcctctctccgagccttccagcccttctttgtggagctcacaatgccttactctgtgattcgtggagaggccttcacactcaaggccacggtcctaaactaccttcccaaatgcatccgggtcagtgtgcagctggaagcctctcccgccttcctagctgtcccagtggagaaggaacaagcgcctcactgcatctgtgcaaacgggcggcaaactgtgtcctgggcagtaaccccaaagtcattaggaaatgtgaatttcactgtgagcgcagaggcactagagtctcaagagctgtgtgggactgaggtgccttcagttcctgaacacggaaggaaagacacagtcatcaagcctctgttggttgaacctgaaggactagagaaggaaacaacattcaactccctactttgtccatcaggtggtgaggtttctgaagaattatccctgaaactgccaccaaatgtggtagaagaatctgcccgagcttctgtctcagttttgggagacatattaggctctgccatgcaaaacacacaaaatcttctccagatgccctatggctgtggagagcagaatatggtcctctttgctcctaacatctatgtactggattatctaaatgaaacacagcagcttactccagagatcaagtccaaggccattggctatctcaacactggttaccagagacagttgaactacaaacactatgatggctcctacagcacctttggggagcgatatggcaggaaccagggcaacacctggctcacagcctttgttctgaagacttttgcccaagctcgagcctacatcttcatcgatgaagcacacattacccaagccctcatatggctctcccagaggcagaaggacaatggctgtttcaggagctctgggtcactgctcaacaatgccataaagggaggagtagaagatgaagtgaccctctccgcctatatcaccatcgcccttctggagattcctctcacagtcactcaccctgttgtccgcaatgccctgttttgcctggagtcagcctggaagacagcacaagaaggggaccatggcagccatgtatataccaaagcactgctggcctatgcttttgccctggcaggtaaccaggacaagaggaaggaagtactcaagtcacttaatgaggaagctgtgaagaaagacaactctgtccattgggagcgccctcagaaacccaaggcaccagtggggcatttttacgaaccccaggctccctctgctgaggtggagatgacatcctatgtgctcctcgcttatctcacggcccagccagccccaacctcggaggacctgacctctgcaaccaacatcgtgaagtggatcacgaagcagcagaatgcccagggcggtttctcctccacccaggacacagtggtggctctccatgctctgtccaaatatggagcagccacatttaccaggactgggaaggctgcacaggtgactatccagtcttcagggacattttccagcaaattccaagtggacaacaacaaccgcctgttactgcagcaggtctcattgccagagctgcctggggaatacagcatgaaagtgacaggagaaggatgtgtctacctccagacatccttgaaatacaatattctcccagaaaaggaagagttcccctttgctttaggagtgcagactctgcctcaaacttgtgatgaacccaaagcccacaccagcttccaaatctccctaagtgtcagttacacagggagccgctctgcctccaacatggcgatcgttgatgtgaagatggtctctggcttcattcccctgaagccaacagtgaaaatgcttgaaagatctaaccatgtgagccggacagaagtcagcagcaaccatgtcttgatttaccttgataaggtgtcaaatcagacactgagcttgttcttcacggttctgcaagatgtcccagtaagagatctgaaaccagccatagtgaaagtctatgattactacgagacggatgagtttgcaattgctgagtacaatgctccttgcagcaaagatcttggaaatgcttgaagaccacaaggctgaaaagtgctttgctggagtcctgttctcagagctccacagaagacacgtgtttttgtatctttaaagacttgatgaataaacactttttctggtcaatgtcaaaaaaaaaaaaaaaaaaaaaaaaa

SEQ ID NO: 3 - protein, 22 aa, Homo sapiens
SLFTDLEAENDVLHCVAFAVPK

SEQ ID NO: 4 - protein, 11 aa, Homo sapiens
AIGYLNTGYQR

SEQ ID NO: 5 - protein, 11 aa, Homo sapiens
LPPNVVEESAR

SEQ ID NO: 6 - protein, 16 aa, Homo sapiens
DTVIKPLLVEPEGLEK

SEQ ID NO: 7 - protein, 10 aa, Homo sapiens
QGIPFFGQVR

SEQ ID NO: 8 - protein, 9 aa, Homo sapiens
FEVQVTVPK

SEQ ID NO: 9 - protein, 13 aa, Homo sapiens
LVHVEEPHTETVR

SEQ ID NO: 10 - protein, 8 aa, Homo sapiens
FQVDNNNR

SEQ ID NO: 11 - protein, 8 aa, Homo sapiens
YGAATFTR

SEQ ID NO: 12 - protein, 8 aa, Homo sapiens
AGLYGLPR

SEQ ID NO: 13 - protein, 11 aa, Homo sapiens
LQVVDQPLPVR

SEQ ID NO: 14 - protein, 7 aa, Homo sapiens
LLEQGLR

SEQ ID NO: 15 - protein, 9 aa, Homo sapiens
GYAVLGGER

SEQ ID NO: 16 - protein, 10 aa, Homo sapiens
GTPLISPLIK

SEQ ID NO: 17 - protein, 9 aa, Homo sapiens
TGTVSLEVR

SEQ ID NO: 18 - protein, 8 aa, Homo sapiens
HGLYLPTR

SEQ ID NO: 19 - protein, 7 aa, Homo sapiens
LYTLQDK

SEQ ID NO: 20 - protein, 10 aa, Homo sapiens
AQVADVVVSR

SEQ ID NO: 21 - protein, 9 aa, Homo sapiens
GLVQALQTK

SEQ ID NO: 22 - protein, 9 aa, Homo sapiens
DPSQQELPR

SEQ ID NO: 23 - protein, 8 aa, Homo sapiens
DGAWGAFR

SEQ ID NO: 24 - protein, 9 aa, Homo sapiens
LSPDAIPGK

SEQ ID NO: 25 - protein, 10 aa, Homo sapiens
VFTTVGSAEK

SEQ ID NO: 26 - protein, 9 aa, Homo sapiens
YSGTLNLDR

SEQ ID NO: 27 - protein, 9 aa, Homo sapiens
ELNLVLSVR

SEQ ID NO: 28 - protein, 7 aa, Homo sapiens
LQELSSK

SEQ ID NO: 29 - protein, 9 aa, Homo sapiens
QVQPEGPYR

SEQ ID NO: 30 - protein, 9 aa, Homo sapiens
VLEALLPLK

SEQ ID NO: 31 - protein, 10 aa, Homo sapiens
VAAAVDLIIK

SEQ ID NO: 32 - protein, 7 aa, Homo sapiens
SHQGLDR

SEQ ID NO: 33 - protein, 8 aa, Homo sapiens
QELSFAAR

SEQ ID NO: 34 - protein, 8 aa, Homo sapiens
AAEQYTPK